(19) United States
(12) Patent Application Publication
(10) Pub. No.: US 2023/0346237 A1
(43) Pub. Date: Nov. 2, 2023

(54) ARTIFICIAL ARTERIES FOR WEARABLE DEVICE CALIBRATION

(71) Applicant: Oura Health Oy, Oulu (FI)

(72) Inventors: Jouni Huopana, Oulu (FI); Jukka-Tapani Mäkinen, Oulu (FI)

(21) Appl. No.: 18/128,932

(22) Filed: Mar. 30, 2023

Related U.S. Application Data
(60) Provisional application No. 63/326,180, filed on Mar. 31, 2022.

(51) Int. Cl. A61B 5/0205; A61B 5/00; A61B 5/026

(52) U.S. Cl. CPC A61B 5/0261; A61B 5/02055; A61B 5/6826

(57) ABSTRACT

Methods, systems, and devices for implementing an artificial artery to calibrate a wearable device are described. An artificial digit may be formed from a material configured with optical properties, thermal properties, or mechanical properties representative of human tissue (e.g., a human finger). The artificial digit may include one or more channels representative of human arteries, capillaries, or both. Additionally, or alternatively, the artificial digit may include electrochromic sheets that may adjust to light absorption properties of the material. A wearable device may be placed on the artificial digit, and one or more sensors of the wearable device may be activated to collect measurements when there is simulated fluid flow through the artificial digit (e.g., via a pump system). The wearable device sensors may be configured, or calibrated, according to the measurements.

### CROSS REFERENCE

The present Application for Patent claims the benefit of U.S. Provisional Pat. Application No. 63/326,180 by HUOPANA et al., entitled “ARTIFICIAL ARTERIES FOR WEARABLE DEVICE CALIBRATION,” filed Mar. 31, 2022, assigned to the assignee hereof and expressly incorporated by reference herein.

### FIELD OF TECHNOLOGY

The following relates to wearable devices and data processing, including artificial arteries for calibration of wearable sensors.

### BACKGROUND

Some wearable devices may be configured to collect data from users, including temperature data, heart rate data, pressure data, motion data, and the like. However, there may be variability between users that may cause inconsistencies in the data. As such, there are technical challenges with calibrating sensors for wearable devices using human tissue or digits.

### BRIEF DESCRIPTION OF THE DRAWINGS

FIG. 1 illustrates an example of a system that supports artificial arteries for wearable device calibration in accordance with aspects of the present disclosure.

FIG. 2 illustrates an example of a system that supports artificial arteries for wearable device calibration in accordance with aspects of the present disclosure.

FIGS. 3A, 3B, and 4 illustrate examples of artificial digit diagrams that support artificial arteries for wearable device calibration in accordance with aspects of the present disclosure.

FIG. 5 illustrates an example of a calibration system that supports artificial arteries for wearable device calibration in accordance with aspects of the present disclosure.

FIG. 6 shows a block diagram of an apparatus that supports artificial arteries for wearable device calibration in accordance with aspects of the present disclosure.

FIG. 7 shows a block diagram of a wearable device manager that supports artificial arteries for wearable device calibration in accordance with aspects of the present disclosure.

FIG. 8 shows a diagram of a system including a device that supports artificial arteries for wearable device calibration in accordance with aspects of the present disclosure.

FIGS. 9 through 13 show flowcharts illustrating methods that support artificial arteries for wearable device calibration in accordance with aspects of the present disclosure.

### DETAILED DESCRIPTION

Some wearable devices may be configured to collect data from users associated with movement and other activities. For example, some wearable devices may be configured to continuously acquire physiological data associated with a user including temperature data, pressure data, heart rate data, and the like. In order to efficiently and accurately track physiological data, a wearable device may be configured to collect data continuously while the user wears the device.

In some cases, physiological data from a wearable device may vary across users according to a type of user, a use case of the wearable device, or the like, which may cause inconsistencies in processing of the data. Thus, one or more wearable device sensors of a wearable device (e.g., a ring, watch, necklace, earring, or any other wearable device placed on a human body part) may be calibrated to accurately collect physiological data. To calibrate a wearable device sensor, similar conditions to the human body may be used. In some cases, human skin, blood, and other components of a human body have varying thicknesses and optical densities, causing variation in measurements from a user which may be difficult to replicate. For example, optical, thermal, and mechanical properties may vary between users or in different use cases (e.g., a cold day or a warm day). Previous testing focuses on static materials, failing to account for the dynamic nature of the human body. A lack of proper calibration may lead to inaccurate or misleading physiological data or a negative experience for the user of the wearable sensor device.

Accordingly, techniques described herein are directed to devices, methods, and systems for calibrating one or more sensors of a wearable device using a device made to represent a human appendage, such as a body component or an artificial digit. In some cases, a finger phantom, which may also be referred to as an artificial digit, may be configured to mimic human tissue and may be in the shape of a human finger. In some other cases, a body component may be configured to mimic human tissue and may be in the shape of any body part (e.g., a human appendage, a human chest, a human neck, a human head, or any other body part). For example, the artificial digit and the body component may be formed from one or more materials with optical, thermal, or mechanical properties that mimic or represent the properties of human tissue. The materials may include polymer materials. The shape of the artificial digit and the body component may scale to different sizes to match different sizes of the wearable device. In some cases, the artificial digit and the body component may have a hollowed space in the center to allow for the insertion of additional material. For example, one or more fluid channels may run through the finger phantom to transport fluid and mimic the effects of arteries, veins, or capillaries. In some cases, a size, location and dimension of fluid channels may vary in humans, and this variation may be controlled with the artificial digit, the body component, or both. In some other cases, the artificial digit, the body component, or both may include sheets of electrochromic material configured to mimic the properties of human skin.

A wearable device with one or more sensors may be placed on the artificial digit, the body component, or both, simulating a user wearing the wearable device sensor, such as a ring. Following placement, the wearable device sensor may be activated and may collect measurements. The measurements may include optical, thermal, or mechanical measurements that indicate physiological data (e.g., temperature, heartbeat, blood oxygen level, which may be referred to as SpO2). In some examples, the artificial digit, the body component, or both may have one or more channels or one or more materials therein to activate the wearable device sensor. For example, the wearable device sensor may be activated when fluid is transported through the phantom finger, to simulate the human circulatory system. The sensors of the wearable device may be calibrated according to the measurements. For example, one or more baseline values may be adjusted to lessen, or prevent, inconsistencies in processing of the physiological data.

In some examples, a system for calibrating the wearable device sensor may include a body component, such as an artificial digit, made of one or more materials with one or more channels (e.g., a fluid channel). Additional apparatuses may be connected to the artificial digit for simulation of human tissue. For example, a pump may be connected to one or more fluid channels (e.g., in a closed loop) to transport fluid. In some cases, fluid properties may be pressure dependent, such that one or more properties of the fluid may change (e.g., viscosity) when pressure is applied to the fluid inside the channel. The pump may be controlled to adjust flow speed, pressure, temperature, and the like to mimic human blood.

The pump may be configured to maintain one or more properties of the artificial digit, the body component, or both. Additionally, or alternatively, a temperature-controlled reservoir may be connected to the artificial digit, the body component, or both (e.g., by one or more channels). In some examples, the temperature-controlled reservoir may be used to change the thermal properties, such as the temperature, of the artificial digit, the body component, or both and simulate human tissue. In some cases, the reservoir may contain two or more fluids that have different optical properties (e.g., optical density) that may be mixed to obtain a dynamic and controllable change of optical properties (e.g., light absorption) to the material travelling in the fluid channels. For example, an SpO2 measurement may be based on a difference in light absorption of blood between oxygenated and non-oxygenated states. In some examples, the system to calibrate a wearable device sensor may include active kinetic platforms to mimic user movement (e.g., running, playing sports, cooking).

Aspects of the disclosure are initially described in the context of systems supporting physiological data collection from users via wearable devices. Additional aspects of the disclosure are described in the context of artificial digit diagrams and calibration systems. Aspects of the disclosure are further illustrated by and described with reference to apparatus diagrams, system diagrams, and flowcharts that relate to artificial arteries.

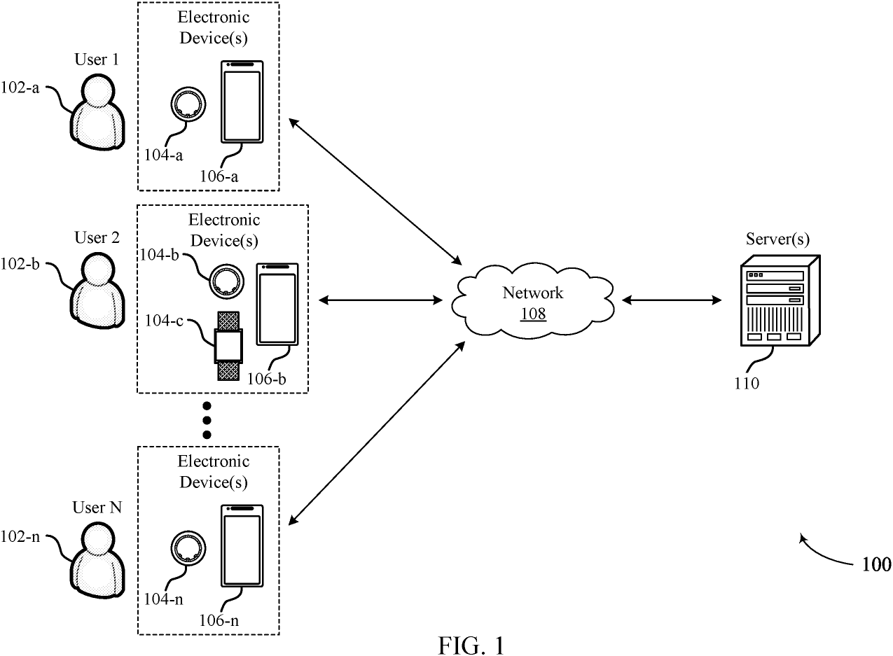

FIG. 1 illustrates an example of a system 100 that supports artificial arteries for wearable device calibration in accordance with aspects of the present disclosure. The system 100 includes a plurality of electronic devices (e.g., wearable devices 104, user devices 106) which may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user’s 102 finger, a wrist computing device (e.g., a smart watch, fitness band, or bracelet) configured to be wom on a user’s 102 wrist, and/or a head mounted computing device (e.g., glasses/goggles). Wearable devices 104 may also include bands, straps (e.g., flexible or inflexible bands or straps), stick-on sensors, and the like, which may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as extemal wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols.

In some implementations, the rings 104 (e.g., wearable devices 104) of the system 100 may be configured to collect physiological data from the respective users 102 based on arterial blood flow within the user’s finger. In particular, a ring 104 may utilize one or more LEDs (e.g., red LEDs, green LEDs) which emit light on the palm-side of a user’s finger to collect physiological data based on arterial blood flow within the user’s finger. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices which utilize LEDs which are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or altemative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time during which a user 102 is asleep, and classify periods of time during which the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time during which the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user’s sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual’s sleep-wake cycle, which repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user’s natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models which are specific to each respective user 102.

In some aspects, the system 100 may utilize other biological rhythms to further improve physiological data collection, analysis, and processing by phase of these other rhythms. For example, if a weekly rhythm is detected within an individual’s baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g. in a hypothetical culture with 12 day “weeks”, 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

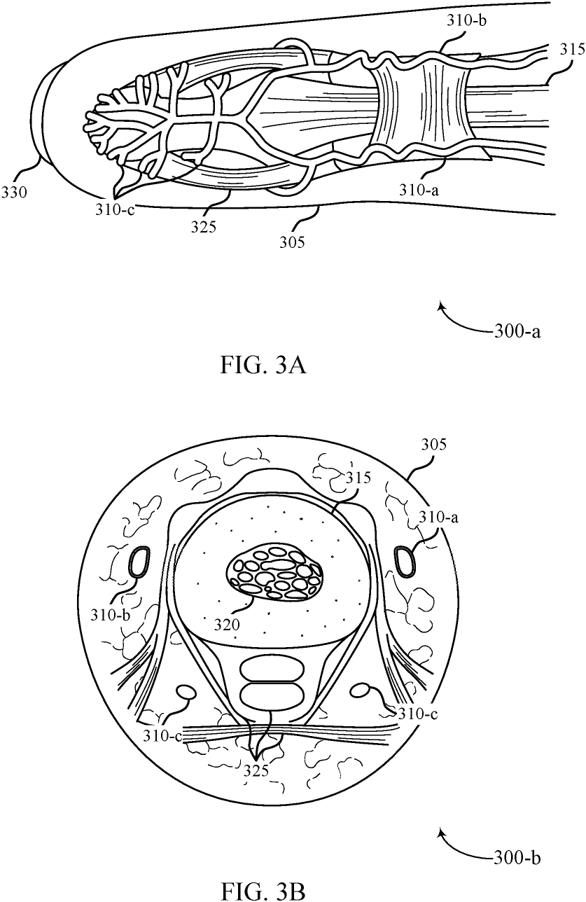

In some aspects, the respective devices of the system 100 may support techniques for implementing an artificial digit to calibrate a wearable device 104 of a user 102. Specifically, techniques described herein support an artificial digit, or phantom finger, made of a polymer material that mimics optical properties, thermal properties, mechanical properties, or any combination thereof of human tissue (e.g., a human finger). For example, the artificial digit may include one or more elements, such as channels representative of human veins (e.g., arteries and capillaries) or electrochromic sheets, liquid crystal layers, a spatial light modulator, or any combination thereof that may adjust light absorption properties of the polymer material, light transmission properties of the polymer material, or the like. The artificial digit may also include one or more artifacts representative of human skin, human ligaments, human bone, or any combination thereof, which is described in further detail with respect to FIG. 3A and FIG. 3B.

In some cases, the artificial digit may be part of a system for calibrating one or more wearable devices. For example, the artificial digit may be connected to a pump, a temperature reservoir, one or more supports for moving the artificial digit through a set of motions, and control electronics. A user 102 of the system may place a wearable device 104 on the artificial digit, and may program fluid to flow through the artificial digit via one or more channels. The channels may move fluid from the pump, through the temperature reservoir, and to the artificial digit. The user 102 may configure the wearable device 104 to take one or more measurements while fluid is flowing through the artificial digit. The wearable device 104 may be configured based on the measurements (e.g., calibrated).

It should be appreciated by a person skilled in the art that one or more aspects of the disclosure may be implemented in a system 100 to additionally, or alternatively, solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

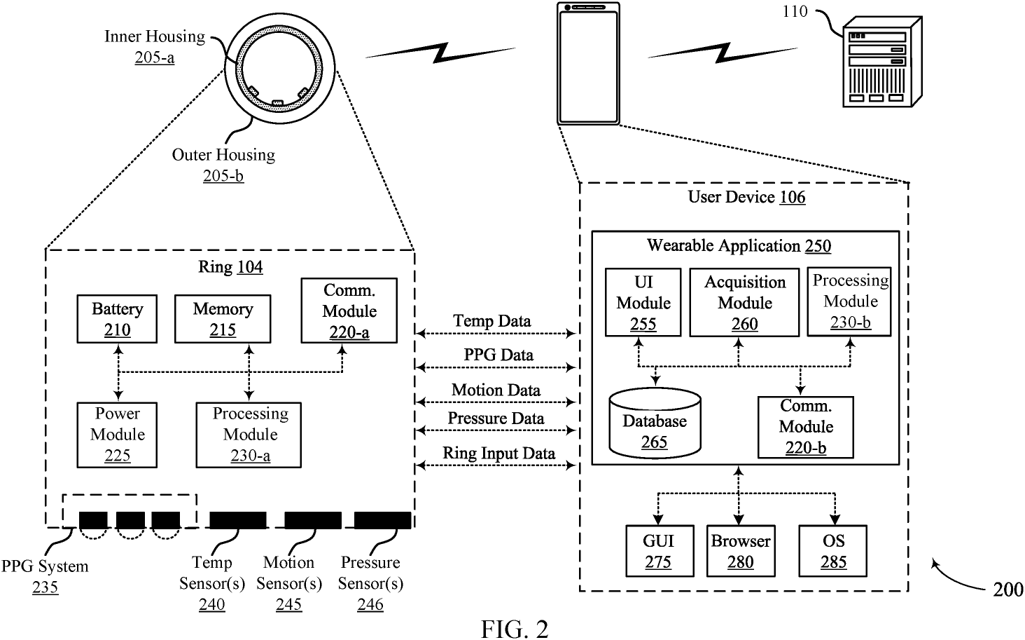

FIG. 2 illustrates an example of a system 200 that supports artificial arteries for wearable device calibration in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user’s finger, and may determine one or more user physiological parameters when worn around the user’s finger. Example measurements and determinations may include, but are not limited to, user skin temperature, pulse waveforms, respiratory rate, heart rate, HRV, blood oxygen levels, and the like.

System 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205, which may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), one or more motion sensors 245, and one or more pressure sensors 246.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components which are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user’s finger (e.g., using a clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, which may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user’s finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, pressure sensors 246, and other sensors) interface with the underside of the user’s finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data, pressure data, or any other data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, pressure data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors which may be used to collect data in addition to, or which supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during 104 charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during 104 charging, and under voltage during 104 discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user’s finger (e.g., skin temperature). In some implementations, the temperature sensor 240 may contact the user’s skin. In other implementations, a portion of the housing 205 (e.g., the inner housing 205-a) may form a barrier (e.g., a thin, thermally conductive barrier) between the temperature sensor 240 and the user’s skin. In some implementations, portions of the ring 104 configured to contact the user’s finger may have thermally conductive portions and thermally insulative portions. The thermally conductive portions may conduct heat from the user’s finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user’s temperature over time. For example, the processing module 230-a may sample the user’s temperature according to a sampling rate. An example sampling rate may include one sample per second, although the processing module 230-a may be configured to sample the temperature signal at other sampling rates that are higher or lower than one sample per second. In some implementations, the processing module 230-a may sample the user’s temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during 104 exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user’s finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user’s finger (e.g., any finger). For example, one or more temperature sensors 240 on the ring 104 may acquire a user’s temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user’s finger may differ from the temperature measured at a user’s wrist or other external body location. Additionally, the distal temperature measured at a user’s finger (e.g., a “shell” temperature) may differ from the user’s core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user’s finger. The PPG signal generated by the PPG system 235 may indicate the perfusion of blood in the illuminated region. For example, the PPG signal may indicate blood volume changes in the illuminated region caused by a user’s pulse pressure. The processing module 230-a may sample the PPG signal and determine a user’s pulse waveform based on the PPG signal. The processing module 230-a may determine a variety of physiological parameters based on the user’s pulse waveform, such as a user’s respiratory rate, heart rate, HRV, oxygen saturation, and other circulatory parameters.

In some implementations, the PPG system 235 may be configured as a reflective PPG system 235 in which the optical receiver(s) receive transmitted light that is reflected through the region of the user’s finger. In some implementations, the PPG system 235 may be configured as a transmissive PPG system 235 in which the optical transmitter(s) and optical receiver(s) are arranged opposite to one another, such that light is transmitted directly through a portion of the user’s finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform, which may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user’s heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBls. The processing module 230-a may store the HRV values over time in the memory 215. Moreover, the processing module 230-a may determine the user’s respiratory rate over time. For example, the processing module 230-a may determine respiratory rate based on frequency modulation, amplitude modulation, or baseline modulation of the user’s IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BM1160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may include one or more pressure sensors 246, such as one or more strain gauges or differential pressure sensors. The pressure sensors 246 may measure a pressure of a liquid (e.g., blood in an artery) relative to atmospheric pressure, or may measure the difference between the pressure of the liquid at different points. For example, the ring 104 may include one or more gauge pressure sensors that indicate blood pressure of a user 102. As another example, the ring 104 may include one or more differential pressure sensors that indicate when a change in pressure has occurred in a user 102. The pressure sensors 246 may be included in one or more sensor packages.

The processing module 230-a may sample the pressure measurements at a sampling rate (e.g., 50 Hz) and determine any changes in pressure at the ring 104 based on the sampled pressure measurements. For example, the processing module 230-a may sample blood pressure to determine a health related anomaly of a user 102. In some implementations, the processing module 230-a may store pressure data in memory 215. Pressure data may include sampled pressure data as well as pressure data that is calculated based on the sampled pressure measurements.

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user’s finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user’s physiological parameters may be measured by sensors included on a ring 104, other devices may measure a user’s physiological parameters. For example, although a user’s temperature may be measured by a temperature sensor 240 included in a ring 104, other devices may measure a user’s temperature. In some examples, other wearable devices (e.g., wrist devices) may include sensors that measure user physiological parameters. Additionally, medical devices, such as external medical devices (e.g., wearable medical devices) and/or implantable medical devices, may measure a user’s physiological parameters. One or more sensors on any type of computing device may be used to implement the techniques described herein.

The physiological measurements may be taken continuously throughout the day and/or night. In some implementations, the physiological measurements may be taken during 104 portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) which may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations which require relatively low processing power and/or operations which require a relatively low latency, whereas the user device 106 may transmit the data to the servers 110 for processing operations which require relatively high processing power and/or operations which may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user’s sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner which is consistent with their sleep schedules. In some cases, users may be able to selectively adjust (e.g., via the GUI) a timing of sleep days relative to calendar days so that the sleep days are aligned with the duration of time in which the respective users typically sleep.

In some implementations, each overall score for a user for each respective day (e.g., Sleep Score, Readiness Score) may be determined/calculated based on one or more “contributors,” “factors,” or “contributing factors.” For example, a user’s overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user’s sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user’s overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user’s needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user’s sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user’s resting heart rate to stabilize during the night. A sign of a very good recovery is that the user’s resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user’s highest temperature during the nap is at least 0.5° C. higher than the highest temperature during the longest period. In some aspects, the ring may measure a user’s body temperature while the user is asleep, and the system 200 may display the user’s average temperature relative to the user’s baseline temperature. If a user’s body temperature is outside of their normal range (e.g., clearly above or below 0.0), the body temperature contributor may be highlighted (e.g., go to a “Pay attention” state) or otherwise generate an alert for the user.

In some aspects, the system 200 may support techniques for calibrating a wearable device using one or more measurements from a body component, such as an artificial digit, or finger phantom. The artificial digit may represent a human finger, such as by having similar optical properties, thermal properties, mechanical properties, or any combination thereof. Additionally, or alternatively, the body component may represent any other human body part, such as an appendage, a neck, a head, a chest, or the like. In some cases, the wearable device may be active while fluid flow through the artificial digit, the body component, or both is simulated. For example, the wearable device may take temperature measurements, one or more PPG measurements, motion measurements, pressure measurements, or any combination thereof using the temperature sensors 240, PPG system 235, motion sensors 245, and pressure sensors 246. In some examples, the PPG measurements may include two separate optical channel PPG measurements to obtain an SpO2 measurement. A user device 106, or another device with access to the data, may use the temperature data, PPG data, motion data, pressure data, or any combination thereof to calibrate the sensors of the wearable device. For example, the sensors may be calibrated to adjust for variability in user-to-user measurements.

FIGS. 3A and 3B illustrate examples of an artificial digit diagram 300-a and an artificial digit diagram 300-b that support artificial arteries for wearable device calibration in accordance with aspects of the present disclosure. The artificial digit diagram 300-a and the artificial digit diagram 300-b may implement, or be implemented by, aspects of the system 100, system 200, or both. For example, the artificial digit diagram 300-a and the artificial digit diagram 300-b may illustrate examples of an artificial digit, which may be an example of a body component, configured to calibrate wearable devices 104 as described with reference to FIG. 1. Specifically, the artificial digit diagram 300-a and the artificial digit diagram 300-b may illustrate the contents and functionality of an artificial digit. Although the artificial digit is illustrated as a finger in FIG. 3A and FIG. 3B, the artificial digit may represent any human body part for any example of a wearable device (e.g., a wrist for a watch, a neck for a necklace, and the like).

In some examples, one or more users, such as users 102 as described with reference to FIG. 1, may use a wearable device to collect one or more health metrics, such as heart rate, body temperature, movement, etc. Each user may have unique attributes, such that an average health metric for each user may vary. For example, a user may have a different average heart rate, body temperature, or the like when compared with another user. Further, the environment of the user may affect the data collected by the wearable device. For example, the time of day, the outdoor temperature, the activities of the user, or any other external factor may affect the measurements of the wearable device. Due to the variations from user-to-user, it may be difficult to use the data from each user to detect health related trends. For example, if a user has a naturally high heartrate, the wearable device may misdiagnose the high heart rate as a trend towards an illness. Similarly, if a different user has a naturally low heartrate, the wearable device may not detect a high heart rate trend towards an illness, instead detecting a heart rate within a “normal” range.

Further, there may be variation in measurement results between wearable devices based on hardware variability. For example, different sensors may produce slightly varying results, which may cause a misdiagnoses of a measurement trend (e.g., similar to user-to-user variability, as described above). It may be difficult to separate the effects of multiple physical and physiological phenomena causing noise in signal measurements of humans (e.g., a human body part, such as human fingers).

Thus, as described herein, the wearable devices may be calibrated using a body component, such as an artificial digit as illustrated in FIG. 3A and FIG. 3B, which may also be referred to as a finger phantom. In some cases, the body component may mimic any human body part, such as a human appendage, a human chest, a human head, a human neck, or the like. For example, the body component may be a human wrist, ankle, arm, leg, finger, or any other appendage to calibrate a wearable device, such as a watch or wrist band, an ankle band, an arm band, a leg band, or a ring, respectively. FIG. 3A shows a top view of the artificial digit, while FIG. 3B shows a cross section of the artificial digit. In some cases, the artificial digit may provide for a user to collect information that may stabilize measurements by the hardware of the wearable device. For example, the artificial digit may simulate one or more conditions that mimic or replicate human equivalents, such that the sensors of the wearable device may be configured according to the results. The artificial digit may replicate one or more properties of human tissue, which may provide a steady platform for reliable hardware measurements.

In some examples, the artificial digit may include a polymer material 305 with optical, thermal, or mechanical properties representative of human tissue. The polymer material 305 may permit a set of wavelengths to penetrate the surface. For example, a white artificial digit may be manufactured for infrared wavelengths and may replicate properties of human skin (e.g., 940 nanometers (nm)), such as for heart rate and blood oxygen level measurements. In some other examples, a black artificial digit may be manufactured to prevent one or more wavelengths from penetrating the polymer material 305 to measure internal stray light in the wearable device sensors. Although the different examples may be referred to as white or black, the visible color of the artificial digit may vary (e.g., may not be white or black), but may still be manufactured to implement the properties described herein. In some examples, the white artificial digits may provide various hardware testing results, while black artificial digits may be used for stray light analysis.

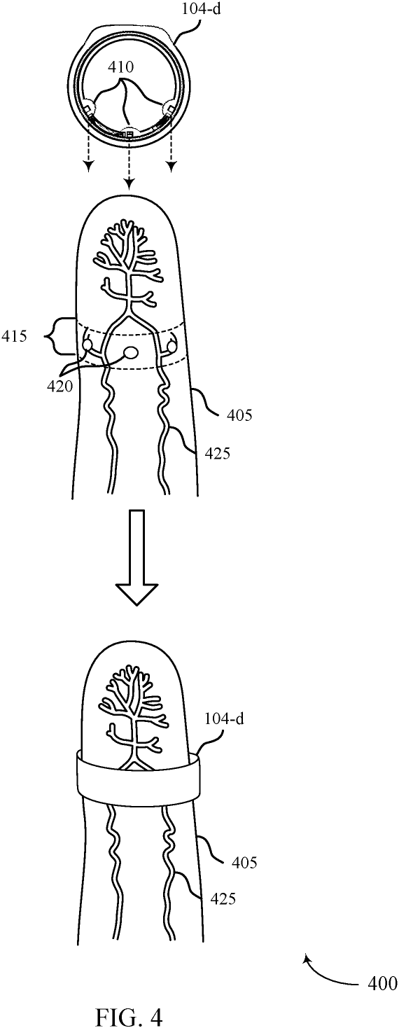

In some cases, the artificial digit may be manufactured to couple with one or more wearable devices, which is described in further detail with respect to FIG. 4. An artificial digit may be designed and produced using a scalable manufacturing process. The manufacturing process may include measuring the wearable device to ensure the circumference of the artificial digit couples directly with one or more sensors of the wearable device. A 3D mold for the artificial digit may be printed or otherwise created. The polymer material 305 may be inserted into the mold to create the artificial digit. The artificial digit may be molded to any size or shape. Similarly, the optical, thermal, and mechanical properties of the artificial digit may be adjusted according to the composition of the polymer material.

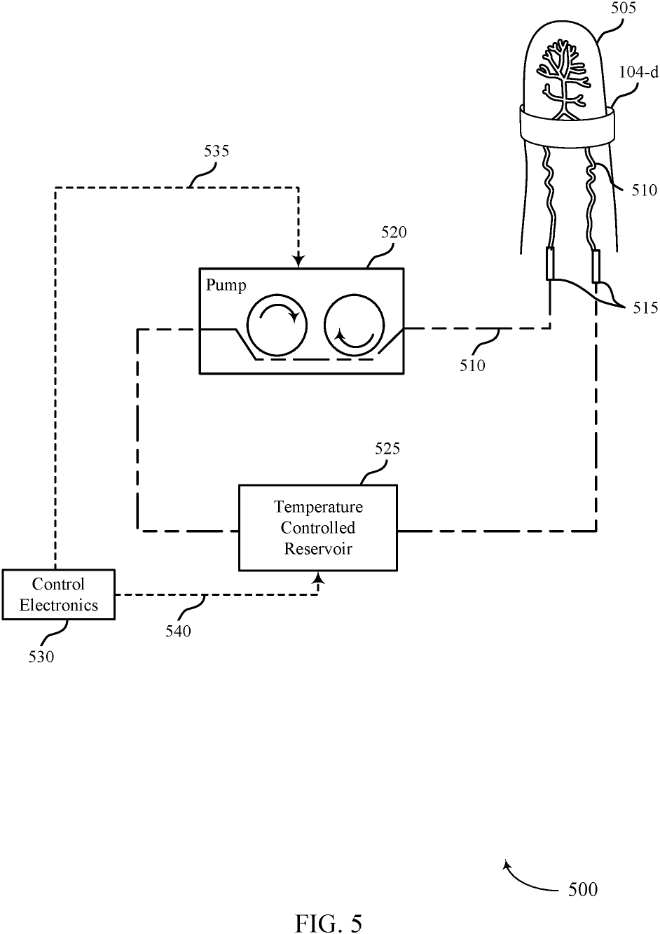

In some examples, the artificial digit may include one or more elements disposed within the polymer material 305. The elements may be configured to simulate fluid flow through the artificial digit. For example, the elements may include one or more channels, such as channel 310-a, channel 310-b, channel 310-c, through which fluid may flow. The channel 310-a and channel 310-b may be configured to simulate fluid flow through one or more arteries in a human extremity, such as a finger. The channel 310-c may be configured to simulate fluid flow through one or more capillaries in the human extremity. Together, channel 310-a, channel 310-b, and channel 310-c, may simulate the flow of blood through veins in a human finger. In some cases, channel 310-a, channel 310-b, channel 310-c, or any combination thereof may be connected to a pump system that provides a constant fluid flow, a variable rate of fluid flow (e.g., simulating a human pulse), or the like, which is described in further detail with respect to FIG. 5.

Each of channel 310-a, channel 310-b, and channel 310-c may be formed by an absence of polymer material 305. Additionally, or alternatively, each of channel 310-a, channel 310-b, and channel 310-c may be hollow tubes inserted into the polymer material that may be made of a different material with properties that mimic the walls of arteries and capillaries in humans (e.g., muscle tissue in the vein walls). The channels may be a threshold distance below the surface of the artificial digit, such that the sensors may collect measurements through the polymer material 305, which may mimic the collection of measurements of blood flow through human skin. For example, the channel 310-a and the channel 310-b, which may represent human arteries, may be located 3 millimeters (mm) below the surface of the artificial digit. The channel 310-a and channel 310-b may have a threshold diameter, such as 1.2 mm. The channel 310-c, which may be representative of human capillaries, may be closer to the surface of the artificial digit than the channel 310-a, the channel 310-b, or both (e.g., 10 microns below the surface). The channel 310-c may branch out from the channel 310-a, the channel 310-b, or both to create layers representative of human veins. Thus, in some examples, a wearable device sensor may test different penetration depths within the polymer material.

In some examples, the elements may include electrochromic sheets, liquid crystal layers, or different types of spatial light modulators configured to adjust light transmission, scatter and absorption properties of the material either through the whole layer at once or selectively on certain spatial locations. In some examples, the absorption properties may be related to a signal from pulsating blood-containing tissue in a PPG measurement. In some cases, an optical parameter may be adjusted locally with one or more spatial light modulator components. Adjusting the local parameter locally may mimic vein pulsations or detailed physiological phenomena. For example, fluid flow through one or more capillary vein structures with pixelated spatial light modulators may be turned on and off to mimic skin vasoconstriction, which may be a physiological phenomenon affecting one or more measurements from sensors of a wearable device (e.g., SpO2 measurements).

The electrochromic sheets, liquid crystal layers, spatial light modulator, or the like may be formed into the shape of the artificial digit, such that a wearable device may be placed on the shape. A user may configure the absorption properties of the electrochromic sheets, liquid crystal layers, spatial light modulator, or the like to represent different properties of human tissue. For example, the user may apply a voltage to the electrochromic sheets, liquid crystal layers, spatial light modulator, or the like to cause one or more reactions. The reactions may create an absorption band at one or more defined wavelengths, which may change the optical properties of the electrochromic sheet. The polymer material 305 together with the channels or the electrochromic sheets, liquid crystal layers, spatial light modulator, or the like may represent optical properties of human skin, such that the wearable device may collect measurements using one or more sensors that may reduce or eliminate user-to-user variability, which is described in further detail with respect to FIG. 4.

In addition to the elements, the artificial digit may include one or more artifacts disposed within the polymer material 305. The artifacts may represent one or more of human bone, human ligaments, human nails, human muscle, or any other aspects of a human appendage (e.g., finger). For example, as illustrated in FIG. 3A and FIG. 3B, the artificial digit may include one or more bone artifacts 315 representative of human bone. Each bone artifact 315 may be made of a material with same optical, thermal, or mechanical properties of human bone, which may include a bone marrow artifact 320. For example, each bone artifact 315 may be made of a material with a stiffness value greater than that of the polymer material 305. In some cases, the bone artifact 315 may include one or more joints that may be configured for a set of motions. For example, the artificial digit may include one or more segments of bone artifacts 315 representative of human joints, each segment able to rotate independently of each other. Additionally, or alternatively, the artificial digit may include one or more ligament artifacts 325 representative of human ligaments, or human tissue. The artificial digit may include a nail artifact 330 representative of human nails. The ligament artifacts 325, the nail artifact 330, or both may be made of a material with same optical, thermal, or mechanical properties of human ligaments or human nails, respectively.

FIG. 4 illustrates an example of an artificial digit diagram 400 that supports artificial arteries for wearable device calibration in accordance with aspects of the present disclosure. The artificial digit diagram 400 may illustrate the coupling of a wearable device 104-d with the artificial digit 405. The wearable device 104-d may be placed on an artificial digit 405 for measurements and sensor calibration. Although the wearable devices are illustrated as rings in FIG. 4, they may be any example of a wearable device (e.g., a watch, a necklace, and the like). Similarly, there may be any number of wearable device sensors 410 in any locations along the wearable device 104-d. The sensors may vary in size and distance from each other. A coupling site 415 may be one or more coupling sites for one or more wearable devices and may be located anywhere on the artificial digit 405.

In some examples, multiple calibrations of multiple of wearable devices may be performed simultaneously. A body component, such as an artificial digit 405, may be any shape or size to match that of the wearable device 104-d and may represent any human body part for any example of a wearable device 104-d (e.g., a wrist for a watch, a neck for a necklace, and the like). Calibration may occur across different wearable device shapes, sizes, colors, and properties.

The wearable device 104-d may couple to the artificial digit 405. Wearable device sensors 410, which may be referred to as sensors, may be located on the inside of the wearable device 104-d. In some examples, the wearable device sensors 410 may be one or more sensors and include LED and photodiode pairs, pressure sensors, thermal sensors, or the like, for detecting optical, thermal, and mechanical properties. The wearable device 104-d may be placed over the artificial digit 405 at the coupling site 415 to mimic a user wearing the wearable device 104-d. In some examples, the coupling site 415 may be a band around the artificial digit 405 of the same width and shape as the wearable device 104-d. The coupling site 415 may have one or more receptors 420 for the wearable device sensors 410. The receptors 420 may match the shape of the wearable device sensors 410 and make full contact with the wearable device sensors 410 to mimic the contact of a wearable device 104-d with the skin of the user. In some examples, full contact of the wearable device sensors 410 with the receptors 420 may reduce variation and improve accuracy of the measurements.

The wearable device sensors 410 may take one or more measurements after activation of the artificial digit 405. For example, activation of the artificial digit 405 may include fluid flow through one or more channels 425, which may cause activation of the wearable device sensors 410 for measurements. In some examples, the rate of flow of the fluid may change, which may be representative of a human pulse. As such, the sensors may measure properties of the fluid and artificial digit 405 (e.g., based on perfusion index) that may be interpreted to indicate vital signs of the user, such as heart rate, blood oxygen level, body temperature, or the like. The sensors may also measure fluid properties correlating to other physiological parameters other than vital signs. For example, turbidity measurements may be used for estimating blood lipid concentrations and this may be mimicked with the artificial digit 405 and the wearable device 104-d for sensor calibration.

The wearable device sensors 410 may include LED sensors which may measure internal stray light within the artificial digit 405 or other properties of the artificial digit 405. In reference to FIG. 2, in some examples, the LED sensors may measure PPG signal data, such as pulse waveforms and data calculated based on the pulse waveforms (e.g., heart rate values, IBI values, HRV values, and respiratory rate values). In some other examples, the LED sensors may measure blood oxygen levels, or SpO2.

The wearable device sensors 410 may include one or more temperature sensors, which may be used to measure the temperature of the artificial digit 405 or temperature of the fluid. For example, temperature of the fluid may mimic body temperature of the user. In some applications, temperature data may be calculated temperature data (e.g., average temperatures). Temperature may affect viscosity of the fluid.

One or more pressure sensors may measure the pressure of the wearable device sensors 410 on the artificial digit 405 (e.g., to detect contact between the sensors and the artificial digit 405). The pressure sensors may be one or more strain gauges or differential pressure sensors. The pressure sensors may detect a loss of coupling if contact between the receptors 420 and the wearable device sensors 410 is not detected. The pressure sensors may measure a variety of properties of the fluid. For example, measurements may include measuring a rate of change of volume of the fluid flowing through the channels 425. As described in reference to FIG. 2, in some examples the pressure sensors may measure the difference in pressure between the fluid and atmosphere, or the fluid at different points. In some other examples, the pressure sensors may indicate blood pressure of the user or a dynamic pressure pulse.

Measurements may be used for calibration of the wearable device sensors 410 and of the wearable device 104-d. In some examples, the wearable device 104-d may collect one or more measurements based on optical, thermal, and mechanical properties of the artificial digit 405. The wearable device 104-d may record these measurements as a baseline, or reference value. The wearable device 104-d may use the reference values when taking measurements of a user of the wearable device 104-d. For example, the wearable device 104-d may compare the measurements of the wearable device sensors 410 from a user to the reference values, and the wearable device sensors 410 may be configured, such as by adjusting an expected value, accordingly. The wearable device sensors 410 may measure temperature of the fluid in the channels 425, and the measured temperature compared to the known temperature of the fluid. In some other examples, the pressure of the fluid, used to mimic blood pressure and heart rate, may be measured and compared to the known pressure to determine how accurate the wearable device sensors 410 are at measuring blood pressure or heart rate. In this manner, the sensors 410 may be calibrated for measurements related to the user experience (e.g., heart rate, blood oxygen level, temperature, blood pressure). For example, internal stray light analysis may be used to improve accuracy of the wearable device sensors 410. Calibration may include the deliberate loss of coupling as detected by the pressure sensors to mimic the possible experiences of the user. In another example, calibration may be used to overcome wavelength variations leading to SpO2 signal measurement errors.

In some examples, calibration of the wearable device 104-d may include coupling a set of supports to the artificial digit 405 and wearable device 104-d to apply a set of movements to the artificial digit 405 while taking various measurements. The set of movements may include movement of the entire artificial digit 405 or may be the movement of segments of the artificial digit 405 (e.g., to mimic the motion of a human body component when performing activities, like playing sports, typing, and other activities). For example, the upper part of the artificial digit 405 may move to mimic an action, such as the act of typing, and measurements of the wearable device sensors 410 may determine if there are changes in blood flow, pressure, and the like, during movement. If contact is lost between the wearable device sensors 410 and the receptors 420, the stray light may dynamically impact measurements by the wearable device sensors 410, which may lead to heart rate or SpO2 measurement error. Thus, calibration may include accounting for user movement and variations in stray light.

FIG. 5 illustrates an example of a calibration system 500 that supports artificial arteries for wearable device calibration in accordance with aspects of the present disclosure. The calibration system 500 may implemented, or be implemented by, aspects of the system 100, system 200, artificial digit diagram 300, artificial digit diagram 400, or a combination thereof. For example, the calibration system 500 may implement an artificial digit 505 for calibrating a wearable device 104 as described with reference to FIGS. 1-4. Although the wearable device 104-d is illustrated as a ring in FIG. 5, aspects and components of the wearable device 104-d illustrated in FIG. 5 may be implemented in any type of wearable device (e.g., a watch, a bracelet, a necklace, and the like).

In some cases, the artificial digit 505 may be an example of a body component, and the calibration system 500 may calibrate any wearable device for a body component. For example, the body component may mimic any human body part, such as a human appendage, a human chest, a human head, a human neck, or the like. The body component may be a human wrist, ankle, arm, leg, finger, or any other appendage to calibrate a wearable device, such as a watch or wrist band, an ankle band, an arm band, a leg band, or a ring, respectively.

A channel 510 may transport one or more fluids through the system 500. The channel 510 may be divided into multiple channels and may enter and exit the artificial digit 505. In some examples, the connectors 515 may provide structure for the channel 510 and fluid to enter and exit the artificial digit 505. The channel 510 may mimic a human arterial structure (e.g., arteries, veins, or capillaries) and replicate the effects of arterial walls on measurements (optical density, temperature, etc.). For example, the channel 510 may be multiple, thinner channels spread throughout the artificial digit 505 to mimic capillaries. In some examples, the fluid may have similar properties to that of blood, such as viscosity and color. The channel 510 may connect to a pump 520.

The pump 520 may facilitate the flow of fluid through the channel 510, which may travel through the artificial digit 505. In some examples, the pump 520 may be a closed loop system that draws the fluid from a temperature controlled reservoir 525 and moves fluid through the channel 510 without returning the fluid to the temperature controlled reservoir 525. In some other examples, the pump 520 may be in an open loop system that draws the fluid from the temperature-controlled reservoir 525 and returns the fluid to the temperature-controlled reservoir 525. In some other examples there may be two or more reservoirs containing liquids or other materials with different optical densities and a mixing system that may be used for adjusting the optical density of the combined fluid pumped through the artificial digit 505. In some examples, the pump may have a set of output parameters (e.g., power, flow rate, pressure, efficiency, and the like) that may be configured by a user. For example, the user may configure a change in the rate of flow according to a frequency to mimic a pulse. From the addition of dynamic flow of the fluid, measurements (e.g., oxygen saturation, SpO2, pressure) may represent the dynamics of a human without some, or all, of the variation. For example, PPG, used to measure heart rate optically, may be measured at the artificial digit 505 as fluid is pumped by the pump 520.

A temperature controlled reservoir 525 may be connected to the pump 520 and the artificial digit 505 by the channel 510. The temperature controlled reservoir 525 may maintain or fluctuate the temperature of the fluid. The adjustment of temperature of the fluid by the temperature controlled reservoir 525 may aid in calibration of the wearable device 104-d. For example, the temperature controlled reservoir 525 may maintain the temperature of the fluid at body temperature to assist in calibrating the wearable device by removing the variation found in the measurements of users. The control electronics 530 may be configured to control the pump 520 via connection 535 and the temperature controlled reservoir 525 via connection 540. Various settings may be configured at the control electronics 530 to change the parameters of the pump 520 or the of the temperature controlled reservoir 525, creating a dynamic yet controlled system for measurements for calibration. In some cases, an environmental temperature of the testing may be configured. For example, an ambient temperature may be changed in addition to the fluid temperature in the artificial digit.

In some examples, the artificial digit 505 or one or more components of the system 500 may be connected to one or more supports or constraints. The supports or constraints may be connected to a kinetic platform. The kinetic platform may move the artificial digit through various series of motions. The motions may mimic the motions of a human (playing sports, grasping objects, typing, etc.) and create variation in measurements such as pressure or temperature to represent the user’s use of the wearable device more accurately.

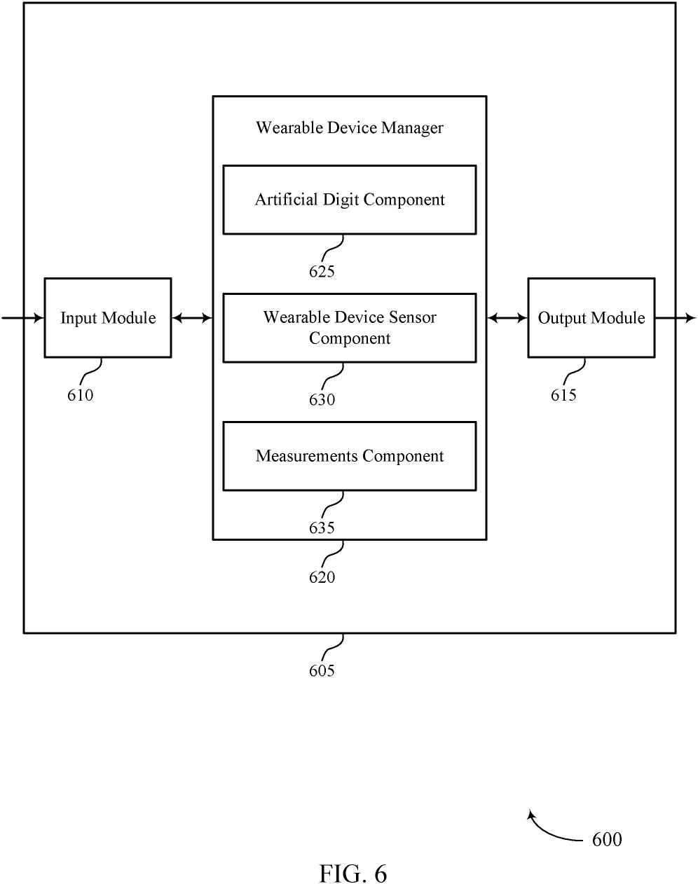

FIG. 6 shows a block diagram 600 of a device 605 that supports artificial arteries for wearable device calibration in accordance with aspects of the present disclosure. The device 605 may include an input module 610, an output module 615, and a wearable device manager 620. The device 605 may also include a processor. Each of these components may be in communication with one another (e.g., via one or more buses).

For example, the wearable device manager 620 may include an artificial digit component 625, a wearable device sensor component 630, a measurements component 635, or any combination thereof. In some examples, the wearable device manager 620, or various components thereof, may be configured to perform various operations (e.g., receiving, monitoring, transmitting) using or otherwise in cooperation with the input module 610, the output module 615, or both. For example, the wearable device manager 620 may receive information from the input module 610, send information to the output module 615, or be integrated in combination with the input module 610, the output module 615, or both to receive information, transmit information, or perform various other operations as described herein.

The wearable device manager 620 may support calibrating a wearable device sensor in accordance with examples as disclosed herein. The artificial digit component 625 may be configured as or otherwise support a means for placing the wearable device sensor on an artificial digit with one or more elements configured to simulate fluid flow through the artificial digit. The wearable device sensor component 630 may be configured as or otherwise support a means for activating the wearable device sensor while the elements are being activated to simulate the fluid flow through the artificial digit. The measurements component 635 may be configured as or otherwise support a means for collecting a plurality of measurements from the artificial digit based at least in part on activating the wearable device sensor.

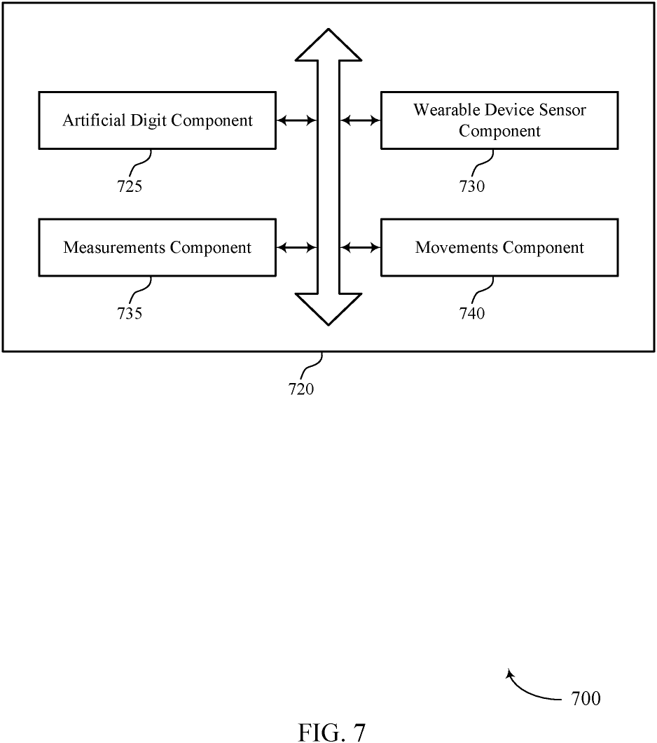

FIG. 7 shows a block diagram 700 of a wearable device manager 720 that supports artificial arteries for wearable device calibration in accordance with aspects of the present disclosure. The wearable device manager 720 may be an example of aspects of a wearable device manager or a wearable device manager 620, or both, as described herein. The wearable device manager 720, or various components thereof, may be an example of means for performing various aspects of artificial arteries for wearable device calibration as described herein. For example, the wearable device manager 720 may include an artificial digit component 725, a wearable device sensor component 730, a measurements component 735, a movements component 740, or any combination thereof. Each of these components may communicate, directly or indirectly, with one another (e.g., via one or more buses).

The wearable device manager 720 may support calibrating a wearable device sensor in accordance with examples as disclosed herein. The artificial digit component 725 may be configured as or otherwise support a means for placing the wearable device sensor on an artificial digit with one or more elements configured to simulate fluid flow through the artificial digit. The wearable device sensor component 730 may be configured as or otherwise support a means for activating the wearable device sensor while the elements are being activated to simulate the fluid flow through the artificial digit. The measurements component 735 may be configured as or otherwise support a means for collecting a plurality of measurements from the artificial digit based at least in part on activating the wearable device sensor.

In some examples, the measurements component 735 may be configured as or otherwise support a means for measuring a rate of change of volume of the fluid through the artificial digit based at least in part on activating the wearable device sensor, wherein the plurality of measurements comprise the rate of change of volume.

In some examples, the measurements component 735 may be configured as or otherwise support a means for measuring a pressure of the wearable sensor on the artificial digit based at least in part on activating the wearable device sensor, wherein the plurality of measurements comprise the pressure.

In some examples, the measurements component 735 may be configured as or otherwise support a means for measuring a temperature of the fluid through the artificial digit based at least in part on activating the wearable device sensor, wherein the plurality of measurements comprise the measured temperature.

In some examples, the wearable device sensor component 730 may be configured as or otherwise support a means for configuring the wearable device sensor based at least in part on analyzing the plurality of measurements.

In some examples, the artificial digit comprises a material that absorbs light in a plurality of wavelengths, and the measurements component 735 may be configured as or otherwise support a means for measuring internal stray light within the artificial digit based at least in part on activating the wearable device sensor, wherein the plurality of measurements comprise the internal stray light.

In some examples, the artificial digit comprises a material that is tuned for one or more wavelengths, and the measurements component 735 may be configured as or otherwise support a means for performing a simulated heart rate measurement, a simulated blood oxygen level, or any combination thereof of the fluid based at least in part on activating the wearable device sensor, wherein the plurality of measurements comprise the simulated heart rate measurement, the simulated blood oxygen level, or any combination thereof.

In some examples, the movements component 740 may be configured as or otherwise support a means for applying a set of movements to the wearable device sensor on the artificial digit, wherein the wearable device sensor is activated during at least one movement of the set of movements.

In some examples, the wearable device sensor comprises one or more of a LED, a pressure sensor, a thermal sensor, or any combination thereof.

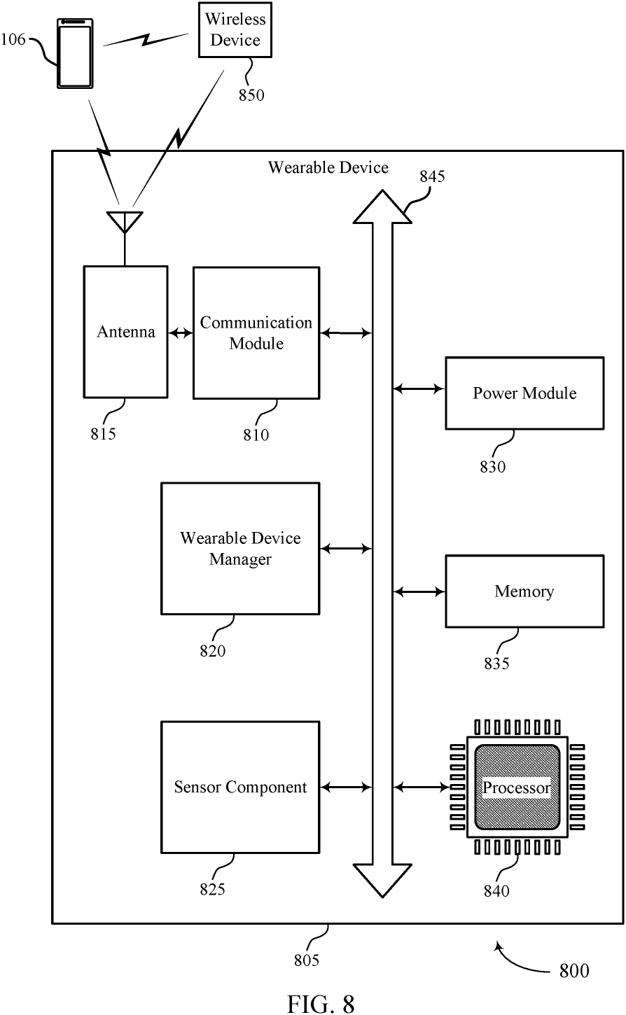

FIG. 8 shows a diagram of a system 800 including a device 805 that supports artificial arteries for wearable device calibration in accordance with aspects of the present disclosure. The device 805 may be an example of or include the components of a device 605 as described herein. The device 805 may include an example of a wearable device 104, as described previously herein. The device 805 may include components for bi-directional communications including components for transmitting and receiving communications with a user device 106 and a server 110, such as a wearable device manager 820, a communication module 810, an antenna 815, a sensor component 825, a power module 830, a memory 835, a processor 840, and a wireless device 850. These components may be in electronic communication or otherwise coupled (e.g., operatively, communicatively, functionally, electronically, electrically) via one or more buses (e.g., a bus 845).

The wearable device manager 820 may support calibrating a wearable device sensor in accordance with examples as disclosed herein. For example, the wearable device manager 820 may be configured as or otherwise support a means for placing the wearable device sensor on an artificial digit with one or more elements configured to simulate fluid flow through the artificial digit. The wearable device manager 820 may be configured as or otherwise support a means for activating the wearable device sensor while the elements are being activated to simulate the fluid flow through the artificial digit. The wearable device manager 820 may be configured as or otherwise support a means for collecting a plurality of measurements from the artificial digit based at least in part on activating the wearable device sensor.

By including or configuring the wearable device manager 820 in accordance with examples as described herein, the device 805 may support techniques for calibrating a wearable device using an artificial digit, which may provide for improved user experience by accounting for variability between users.

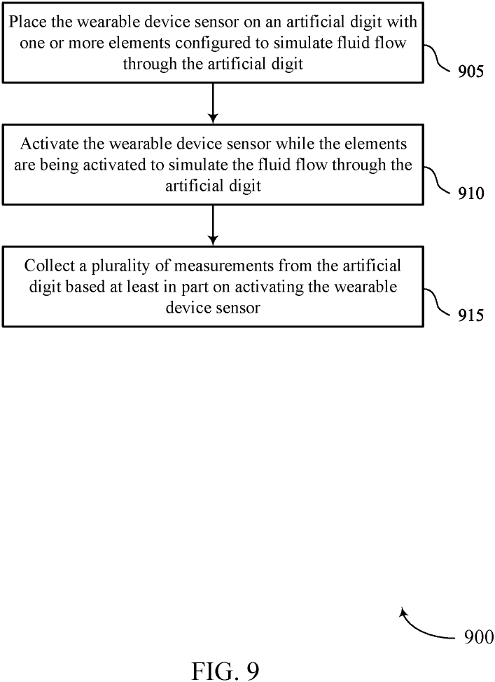

FIG. 9 shows a flowchart illustrating a method 900 that supports artificial arteries for wearable device calibration in accordance with aspects of the present disclosure. The operations of the method 900 may be implemented by a wearable device or its components as described herein. For example, the operations of the method 900 may be performed by a wearable device as described with reference to FIGS. 1 through 8. In some examples, a wearable device may execute a set of instructions to control the functional elements of the wearable device to perform the described functions. Additionally, or alternatively, the wearable device may perform aspects of the described functions using special-purpose hardware.

At 905, the method may include placing the wearable device sensor on an artificial digit with one or more elements configured to simulate fluid flow through the artificial digit. The operations of 905 may be performed in accordance with examples as disclosed herein. In some examples, aspects of the operations of 905 may be performed by an artificial digit component 725 as described with reference to FIG. 7.

At 910, the method may include activating the wearable device sensor while the elements are being activated to simulate the fluid flow through the artificial digit. The operations of 910 may be performed in accordance with examples as disclosed herein. In some examples, aspects of the operations of 910 may be performed by a wearable device sensor component 730 as described with reference to FIG. 7.

At 915, the method may include collecting a plurality of measurements from the artificial digit based at least in part on activating the wearable device sensor. The operations of 915 may be performed in accordance with examples as disclosed herein. In some examples, aspects of the operations of 915 may be performed by a measurements component 735 as described with reference to FIG. 7.

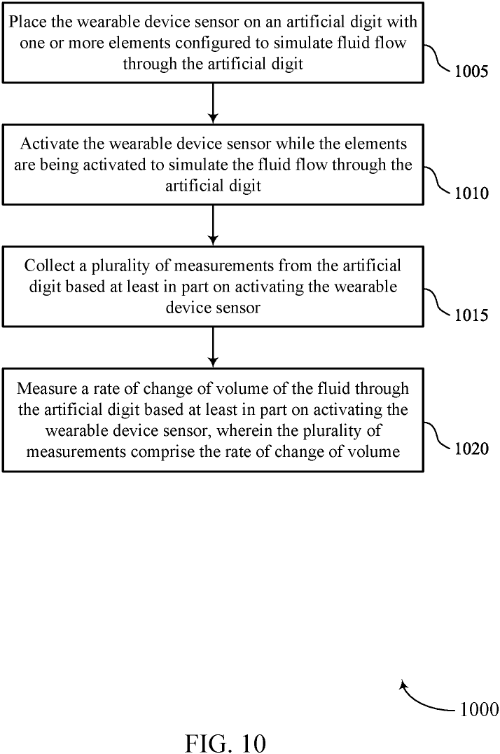

FIG. 10 shows a flowchart illustrating a method 1000 that supports artificial arteries for wearable device calibration in accordance with aspects of the present disclosure. The operations of the method 1000 may be implemented by a wearable device or its components as described herein. For example, the operations of the method 1000 may be performed by a wearable device as described with reference to FIGS. 1 through 8. In some examples, a wearable device may execute a set of instructions to control the functional elements of the wearable device to perform the described functions. Additionally, or alternatively, the wearable device may perform aspects of the described functions using special-purpose hardware.

At 1005, the method may include placing the wearable device sensor on an artificial digit with one or more elements configured to simulate fluid flow through the artificial digit. The operations of 1005 may be performed in accordance with examples as disclosed herein. In some examples, aspects of the operations of 1005 may be performed by an artificial digit component 725 as described with reference to FIG. 7.

At 1010, the method may include activating the wearable device sensor while the elements are being activated to simulate the fluid flow through the artificial digit. The operations of 1010 may be performed in accordance with examples as disclosed herein. In some examples, aspects of the operations of 1010 may be performed by a wearable device sensor component 730 as described with reference to FIG. 7.

At 1015, the method may include collecting a plurality of measurements from the artificial digit based at least in part on activating the wearable device sensor. The operations of 1015 may be performed in accordance with examples as disclosed herein. In some examples, aspects of the operations of 1015 may be performed by a measurements component 735 as described with reference to FIG. 7.

At 1020, the method may include measuring a rate of change of volume of the fluid through the artificial digit based at least in part on activating the wearable device sensor, wherein the plurality of measurements comprise the rate of change of volume. The operations of 1020 may be performed in accordance with examples as disclosed herein. In some examples, aspects of the operations of 1020 may be performed by a measurements component 735 as described with reference to FIG. 7.

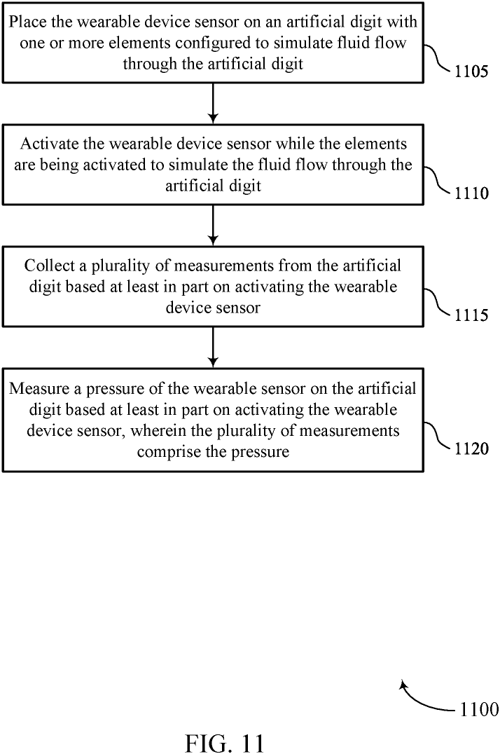

FIG. 11 shows a flowchart illustrating a method 1100 that supports artificial arteries for wearable device calibration in accordance with aspects of the present disclosure. The operations of the method 1100 may be implemented by a wearable device or its components as described herein. For example, the operations of the method 1100 may be performed by a wearable device as described with reference to FIGS. 1 through 8. In some examples, a wearable device may execute a set of instructions to control the functional elements of the wearable device to perform the described functions. Additionally, or alternatively, the wearable device may perform aspects of the described functions using special-purpose hardware.

At 1105, the method may include placing the wearable device sensor on an artificial digit with one or more elements configured to simulate fluid flow through the artificial digit. The operations of 1105 may be performed in accordance with examples as disclosed herein. In some examples, aspects of the operations of 1105 may be performed by an artificial digit component 725 as described with reference to FIG. 7.

At 1110, the method may include activating the wearable device sensor while the elements are being activated to simulate the fluid flow through the artificial digit. The operations of 1110 may be performed in accordance with examples as disclosed herein. In some examples, aspects of the operations of 1110 may be performed by a wearable device sensor component 730 as described with reference to FIG. 7.

At 1115, the method may include collecting a plurality of measurements from the artificial digit based at least in part on activating the wearable device sensor. The operations of 1115 may be performed in accordance with examples as disclosed herein. In some examples, aspects of the operations of 1115 may be performed by a measurements component 735 as described with reference to FIG. 7.

At 1120, the method may include measuring a pressure of the wearable sensor on the artificial digit based at least in part on activating the wearable device sensor, wherein the plurality of measurements comprise the pressure. The operations of 1120 may be performed in accordance with examples as disclosed herein. In some examples, aspects of the operations of 1120 may be performed by a measurements component 735 as described with reference to FIG. 7.

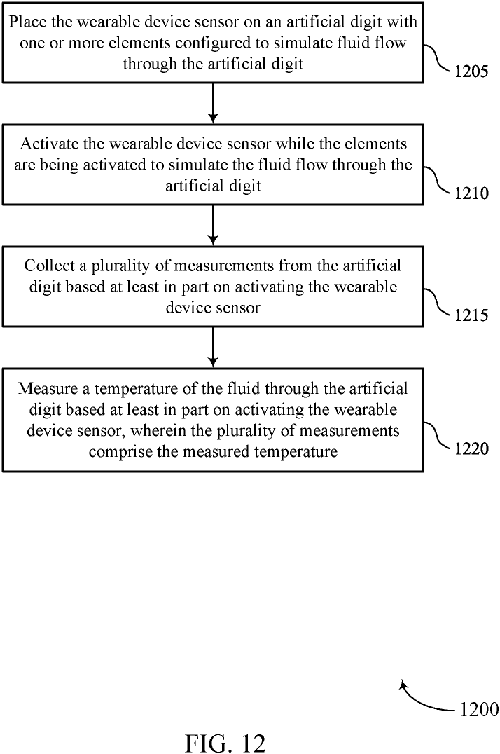

FIG. 12 shows a flowchart illustrating a method 1200 that supports artificial arteries for wearable device calibration in accordance with aspects of the present disclosure. The operations of the method 1200 may be implemented by a wearable device or its components as described herein. For example, the operations of the method 1200 may be performed by a wearable device as described with reference to FIGS. 1 through 8. In some examples, a wearable device may execute a set of instructions to control the functional elements of the wearable device to perform the described functions. Additionally, or alternatively, the wearable device may perform aspects of the described functions using special-purpose hardware.

At 1205, the method may include placing the wearable device sensor on an artificial digit with one or more elements configured to simulate fluid flow through the artificial digit. The operations of 1205 may be performed in accordance with examples as disclosed herein. In some examples, aspects of the operations of 1205 may be performed by an artificial digit component 725 as described with reference to FIG. 7.

At 1210, the method may include activating the wearable device sensor while the elements are being activated to simulate the fluid flow through the artificial digit. The operations of 1210 may be performed in accordance with examples as disclosed herein. In some examples, aspects of the operations of 1210 may be performed by a wearable device sensor component 730 as described with reference to FIG. 7.

At 1215, the method may include collecting a plurality of measurements from the artificial digit based at least in part on activating the wearable device sensor. The operations of 1215 may be performed in accordance with examples as disclosed herein. In some examples, aspects of the operations of 1215 may be performed by a measurements component 735 as described with reference to FIG. 7.

At 1220, the method may include measuring a temperature of the fluid through the artificial digit based at least in part on activating the wearable device sensor, wherein the plurality of measurements comprise the measured temperature. The operations of 1220 may be performed in accordance with examples as disclosed herein. In some examples, aspects of the operations of 1220 may be performed by a measurements component 735 as described with reference to FIG. 7.

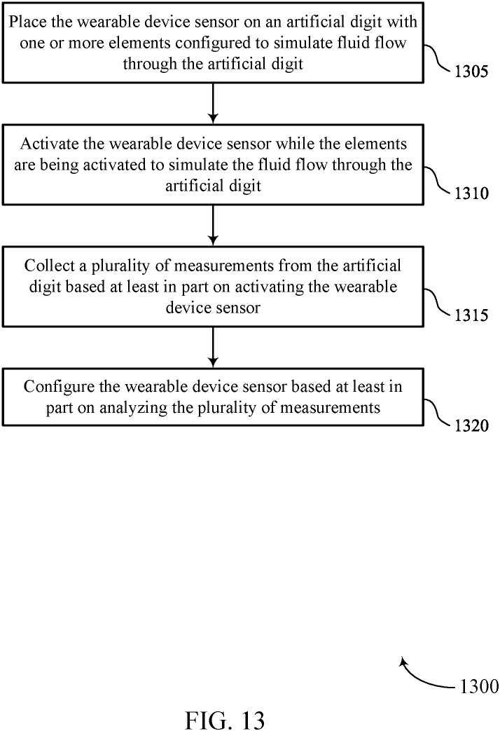

FIG. 13 shows a flowchart illustrating a method 1300 that supports artificial arteries for wearable device calibration in accordance with aspects of the present disclosure. The operations of the method 1300 may be implemented by a wearable device or its components as described herein. For example, the operations of the method 1300 may be performed by a wearable device as described with reference to FIGS. 1 through 8. In some examples, a wearable device may execute a set of instructions to control the functional elements of the wearable device to perform the described functions. Additionally, or alternatively, the wearable device may perform aspects of the described functions using special-purpose hardware.

At 1305, the method may include placing the wearable device sensor on an artificial digit with one or more elements configured to simulate fluid flow through the artificial digit. The operations of 1305 may be performed in accordance with examples as disclosed herein. In some examples, aspects of the operations of 1305 may be performed by an artificial digit component 725 as described with reference to FIG. 7.

At 1310, the method may include activating the wearable device sensor while the elements are being activated to simulate the fluid flow through the artificial digit. The operations of 1310 may be performed in accordance with examples as disclosed herein. In some examples, aspects of the operations of 1310 may be performed by a wearable device sensor component 730 as described with reference to FIG. 7.

At 1315, the method may include collecting a plurality of measurements from the artificial digit based at least in part on activating the wearable device sensor. The operations of 1315 may be performed in accordance with examples as disclosed herein. In some examples, aspects of the operations of 1315 may be performed by a measurements component 735 as described with reference to FIG. 7.

At 1320, the method may include configuring the wearable device sensor based at least in part on analyzing the plurality of measurements. The operations of 1320 may be performed in accordance with examples as disclosed herein. In some examples, aspects of the operations of 1320 may be performed by a wearable device sensor component 730 as described with reference to FIG. 7.

It should be noted that the methods described above describe possible implementations, and that the operations and the steps may be rearranged or otherwise modified and that other implementations are possible. Furthermore, aspects from two or more of the methods may be combined.

An apparatus for calibrating a wearable device sensor is described. The apparatus may include an artificial digit formed from at least a first material configured with one or more of optical properties, thermal properties, or mechanical properties representative of human tissue and one or more elements disposed within the first material, the one or more elements configured to simulate fluid flow through the artificial digit.

In some examples of the apparatuses described herein, the one or more elements include fluid channels configured to transport fluid through the artificial digit.

In some examples of the apparatuses described herein, the one or more fluid channels are further configured to adjust a rate of change of fluid flow through the artificial digit to mimic a human pulse.

In some examples of the apparatuses described herein, the one or more fluid channels are configured to represent a size and location of one or more human arteries within the artificial digit, one or more human capillaries within the artificial digit, or any combination thereof.

In some examples of the apparatuses described herein, the one or more elements include electrochromic sheets, liquid crystal layers, spatial light modulator, or the like configured to adjust light absorption properties, light transmission properties, or both of the material.

In some examples of the apparatus described herein, the artificial digit includes one or more artifacts disposed within the first material including one or more of the optical properties, the thermal properties, or the mechanical properties representative of human bone, human muscle, or any combination thereof.

In some examples of the apparatuses described herein, the first material is a polymer material representative of human skin tissue.

In some examples of the apparatuses described herein, the optical properties include scattering properties, absorption properties, or any combination thereof.

In some examples of the apparatuses described herein, the thermal properties include a temperature of the simulated fluid flow, a rate of change of the temperature, or any combination thereof.

In some examples of the apparatuses described herein, the mechanical properties include a range of motion of the artificial digit.

In some examples of the apparatuses described herein, the artificial digit is in a shape representative of a human finger.

A system for calibrating a wearable device sensor is described. The system may include an artificial digit with one or more fluid channels configured to transport fluid through the artificial digit and a pump connected to the one or more fluid channels and configured to cause the fluid to flow through the one or more fluid channels.

In some examples of the apparatus described herein, the system includes one or more temperature controlled reservoirs and a mixing system connected between the one or more fluid channels, where the pump is configured to maintain a set temperature of the fluid.

In some examples of the system described herein, the pump is further configured to adjust a rate of flow of the fluid through the one or more fluid channels.

In some examples of the apparatus described herein, the system includes a movable support including one or more axis of motion, where the movable support is configured to move the artificial digit according to a set of movements within a movement range.

A method for calibrating a wearable device sensor is described. The method may include placing the wearable device sensor on an artificial digit with one or more elements configured to simulate fluid flow through the artificial digit, activating the wearable device sensor while the elements are being activated to simulate the fluid flow through the artificial digit, and collecting a plurality of measurements from the artificial digit based at least in part on activating the wearable device sensor.

An apparatus for calibrating a wearable device sensor is described. The apparatus may include means for placing the wearable device sensor on an artificial digit with one or more elements configured to simulate fluid flow through the artificial digit, means for activating the wearable device sensor while the elements are being activated to simulate the fluid flow through the artificial digit, and means for collecting a plurality of measurements from the artificial digit based at least in part on activating the wearable device sensor.

A non-transitory computer-readable medium storing code for calibrating a wearable device sensor is described. The code may include instructions executable by a processor to place the wearable device sensor on an artificial digit with one or more elements configured to simulate fluid flow through the artificial digit, activate the wearable device sensor while the elements are being activated to simulate the fluid flow through the artificial digit, and collect a plurality of measurements from the artificial digit based at least in part on activating the wearable device sensor.

Some examples of the method, apparatuses, and non-transitory computer-readable medium described herein may further include operations, features, means, or instructions for measuring a rate of change of volume of the fluid through the artificial digit based at least in part on activating the wearable device sensor, where the plurality of measurements include the rate of change of volume.

Some examples of the method, apparatuses, and non-transitory computer-readable medium described herein may further include operations, features, means, or instructions for measuring a pressure of the wearable sensor on the artificial digit based at least in part on activating the wearable device sensor, where the plurality of measurements include the pressure.

Some examples of the method, apparatuses, and non-transitory computer-readable medium described herein may further include operations, features, means, or instructions for measuring a temperature of the fluid through the artificial digit based at least in part on activating the wearable device sensor, where the plurality of measurements include the measured temperature.

Some examples of the method, apparatuses, and non-transitory computer-readable medium described herein may further include operations, features, means, or instructions for configuring the wearable device sensor based at least in part on analyzing the plurality of measurements.

In some examples of the method, apparatuses, and non-transitory computer-readable medium described herein, the artificial digit includes a material that absorbs light in a plurality of wavelengths and the method, apparatuses, and non-transitory computer-readable medium may include further operations, features, means, or instructions for measuring internal stray light within the artificial digit based at least in part on activating the wearable device sensor, where the plurality of measurements include the internal stray light.

In some examples of the method, apparatuses, and non-transitory computer-readable medium described herein, the artificial digit includes a material that may be tuned for one or more wavelengths and the method, apparatuses, and non-transitory computer-readable medium may include further operations, features, means, or instructions for performing a simulated heart rate measurement, a simulated blood oxygen level, or any combination thereof of the fluid based at least in part on activating the wearable device sensor, where the plurality of measurements include the simulated heart rate measurement, the simulated blood oxygen level, or any combination thereof.

Some examples of the method, apparatuses, and non-transitory computer-readable medium described herein may further include operations, features, means, or instructions for applying a set of movements to the wearable device sensor on the artificial digit, where the wearable device sensor may be activated during at least one movement of the set of movements.

In some examples of the method, apparatuses, and non-transitory computer-readable medium described herein, the wearable device sensor includes one or more of a LED, a pressure sensor, a thermal sensor, or any combination thereof.

An apparatus for calibrating a wearable device sensor is described. The apparatus may include a body component formed from at least a first material configured with one or more of optical properties representative of human tissue and one or more elements disposed within the first material, the one or more elements configured to simulate fluid flow through the body component.

In some examples of the apparatuses described herein, the one or more elements include fluid channels configured to transport fluid through the body component.

In some examples of the apparatuses described herein, the one or more fluid channels are further configured to adjust a rate of change of fluid flow through the body component to mimic a human pulse.

In some examples of the apparatuses described herein, the one or more fluid channels are configured to represent a size and location of one or more human arteries within the body component, one or more human capillaries within the body component, or any combination thereof.

In some examples of the apparatuses described herein, the one or more elements include electrochromic sheets, liquid crystal layers, spatial light modulator, or the like configured to adjust light absorption properties, light transmission properties, or both of the material.

In some examples of the apparatus described herein, the body component includes one or more artifacts disposed within the first material including the optical properties representative of human bone, human muscle, or any combination thereof.

In some examples of the apparatuses described herein, the first material is a polymer material representative of human skin tissue.

In some examples of the apparatuses described herein, the optical properties include scattering properties, absorption properties, or any combination thereof.

In some examples of the apparatuses described herein, the body component is in a shape representative of a human appendage, a human chest, a human head, or a human neck.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A device for calibrating a wearable device sensor, comprising:
an artificial digit formed from at least a first material configured with one or more of optical properties, thermal properties, or mechanical properties representative of human tissue; and
one or more elements disposed within the first material, the one or more elements configured to simulate fluid flow through the artificial digit.

2. The device of claim 1, wherein the one or more elements comprise one or more fluid channels configured to transport fluid through the artificial digit.

3. The device of claim 2, wherein the one or more fluid channels are further configured to adjust a rate of change of fluid flow through the artificial digit to mimic a human pulse.

4. The device of claim 2, wherein the one or more fluid channels are configured to represent a size and location of one or more human arteries within the artificial digit, one or more human capillaries within the artificial digit, or any combination thereof.

5. The device of claim 1, wherein the one or more elements comprise electrochromic sheets, liquid crystal layers, a spatial light modulator, or any combination thereof configured to adjust light absorption properties, light transmission properties, or both of the first material.

6. The device of claim 1, further comprising:
one or more artifacts disposed within the first material comprising one or more of the optical properties, the thermal properties, or the mechanical properties representative of human bone, human muscle, or any combination thereof.

7. The device of claim 1, wherein the first material is a polymer material representative of human skin tissue.

8. The device of claim 1, wherein the optical properties comprise scattering properties, absorption properties, or any combination thereof.

9. The device of claim 1, wherein the thermal properties comprise a temperature of the simulated fluid flow, a rate of change of the temperature, or any combination thereof.

10. The device of claim 1, wherein the mechanical properties comprise a range of motion of the artificial digit.

11. The device of claim 1, wherein the artificial digit is in a shape representative of a human finger.

12. A system for calibrating a wearable device sensor, comprising:
an artificial digit with one or more fluid channels configured to transport fluid through the artificial digit; and
a pump connected to the one or more fluid channels and configured to cause the fluid to flow through the one or more fluid channels.

13. The system of claim 12, further comprising:
one or more temperature controlled reservoirs and a mixing system connected between the one or more fluid channels, wherein the pump is configured to maintain a set temperature of the fluid.

14. The system of claim 12, wherein the pump is further configured to adjust a rate of flow of the fluid through the one or more fluid channels.

15. The system of claim 12, further comprising:
a movable support comprising one or more axis of motion, wherein the movable support is configured to move the artificial digit according to a set of movements within a movement range.

16. A method for calibrating a wearable device sensor, comprising:
placing the wearable device sensor on an artificial digit with one or more elements configured to simulate fluid flow through the artificial digit;
activating the wearable device sensor while the one or more elements are being activated to simulate the fluid flow through the artificial digit; and
collecting a plurality of measurements from the artificial digit based at least in part on activating the wearable device sensor.

17. The method of claim 16, further comprising:
measuring a rate of change of volume of the fluid flow through the artificial digit based at least in part on activating the wearable device sensor, wherein the plurality of measurements comprise the rate of change of volume.

18. The method of claim 16, further comprising:
measuring a pressure of the wearable device sensor on the artificial digit based at least in part on activating the wearable device sensor, wherein the plurality of measurements comprise the pressure.

19. The method of claim 16, further comprising:
measuring a temperature of the fluid flow through the artificial digit based at least in part on activating the wearable device sensor, wherein the plurality of measurements comprise the measured temperature.

20. The method of claim 16, further comprising:
configuring the wearable device sensor based at least in part on analyzing the plurality of measurements.

21. The method of claim 16, wherein the artificial digit comprises a material that absorbs light in a plurality of wavelengths, the method further comprising:
measuring internal stray light within the artificial digit based at least in part on activating the wearable device sensor, wherein the plurality of measurements comprise the internal stray light.

22. The method of claim 16, wherein the artificial digit comprises a material that is tuned for one or more wavelengths, the method further comprising:
performing a simulated heart rate measurement, a simulated blood oxygen level, or any combination thereof of the fluid flow based at least in part on activating the wearable device sensor, wherein the plurality of measurements comprise the simulated heart rate measurement, the simulated blood oxygen level, or any combination thereof.

23. The method of claim 16, further comprising:
applying a set of movements to the wearable device sensor on the artificial digit, wherein the wearable device sensor is activated during at least one movement of the set of movements.

24. The method of claim 16, wherein the wearable device sensor comprises one or more of a light emitting diode, a pressure sensor, a thermal sensor, or any combination thereof.

25. A device for calibrating a wearable device sensor, comprising:
a body component formed from at least a first material configured with one or more of optical properties representative of human tissue; and
one or more elements disposed within the first material, the one or more elements configured to simulate fluid flow through the body component.

26. The device of claim 25, wherein the one or more elements comprise one or more fluid channels configured to transport fluid through the body component.

27. The device of claim 26, wherein the one or more fluid channels are further configured to adjust a rate of change of fluid flow through the body component to mimic a human pulse.

28. The device of claim 26, wherein the one or more fluid channels are configured to represent a size and location of one or more human arteries within the body component, one or more human capillaries within the body component, or any combination thereof.

29. The device of claim 25, wherein the one or more elements comprise electrochromic sheets, liquid crystal layers, a spatial light modulator, or any combination thereof configured to adjust light absorption properties, light transmission properties, or both of the first material.

30. The device of claim 25, further comprising:
one or more artifacts disposed within the first material comprising the optical properties representative of human bone, human muscle, or any combination thereof.

31. The device of claim 25, wherein the first material is a polymer material representative of human skin tissue.

32. The device of claim 25, wherein the optical properties comprise scattering properties, absorption properties, or any combination thereof.

33. The device of claim 25, wherein the body component is in a shape representative of a human appendage, a human chest, a human head, or a human neck.
